(19) United States
(12) Patent Application Publication
(10) Pub. No.: US 2012/0171769 A1
(43) Pub. Date: Jul. 5, 2012

(54) CELL ATTACHMENT COATINGS AND METHODS

(76) Inventors: Joseph S. McGonigle, Minnetonka, MN (US); Jeff J. Ross, Minneapolis, MN (US)

(21) Appl. No.: 13/341,208

(22) Filed: Dec. 30, 2011

Related U.S. Application Data
(60) Provisional application No. 61/428,343, filed on Dec. 30, 2010.

(51) Int. Cl. C12N 5/071; B32B 27/08; B05D 3/06; B32B 27/34

(52) U.S. Cl. 435/402; 428/474.4; 428/336; 427/508

(57) ABSTRACT

Cell attachment coatings for articles such as implantable medical devices and cell culture vessels are disclosed. The coatings include an intermediate coater layer which includes a sulfonated component that is bonded in the coating by reacted aryl ketone functional groups. The coating also include a second coated layer including an immobilized ECM protein or peptide that includes an active portion of an ECM protein that is able to serve as an outer layer to contact cells during use. The coatings promoted enhanced cell binding and growth.

### PRIORITY CLAIM

This application claims the benefit of U.S. Provisional Patent Application Ser. No. 61/428,343 filed Dec. 30, 2010, entitled CELL ATTACHMENT COATINGS AND METHODS, the disclosure of which is incorporated herein by reference.

### FIELD OF THE INVENTION

The invention relates to cell attachment coatings for articles and methods for enhancing cell attachment to an article surface.

### BACKGROUND OF THE INVENTION

Thin surface coatings on implantable medical articles have proved to be valuable in cases where it is desired to provide the article surface with a property that is not present on the uncoated surface. Polymeric coatings have been used to improve the wettability and lubricity of surfaces, and have also been used to present or elute drugs. For example, drugs presented on, or delivered from, the article surface can locally or systemically affect blood and vascular components thereby affecting bodily processes such as hemostasis and angiogenesis.

It has become appreciated that function of the implanted device at the site of implantation can be greatly enhanced by improving its compatibility in the context of the tissue response that occurs as a result of the implantation. Ideally, improved compatibility would allow surfaces of the implanted device to mimic natural tissue exposed by an injury and provide an environment for the formation of normal tissue as a result of the healing process. Polymeric coatings have been applied to surfaces of implantable devices in attempts to promote such tissue formation following implantation. Such surfaces would ideally attract components such as cells to the surface of the device and also promote proliferation of the cells for the formation of tissue.

Some polymeric coatings have been prepared using extracellular matrix proteins such as collagen as a coating material in attempts to attract cells to promote tissue growth on the coated surface. In the body, collagens have been shown to interact with various proteins including von Willebrand factor (VWF), integrins, and bone growth proteins. The direct or indirect result of these interactions can affect cell attachment and tissue formation. However, the process of mimicking the natural function of collagen on a synthetic surface is technically challenging. Preparation of collagen-containing coatings can often result in surfaces that do not provide the intended function following implantation. In coatings wherein collagen is not properly immobilized, collagen can leach out or be released from the surface, rendering the surface ineffective. Also, some chemistries for covalent immobilization of collagen may reduce or destroy collagen activity, such as by altering peptide motifs that are important for the interaction of collagen with other biological components. Certain chemistries may also alter the macromolecular configuration of collagen so that it does not resemble natural collagen. Further, even if collagen is successfully immobilized in a coating, the coating may have the ability to attract cells to a certain extent, but not in a manner that provides for subsequent proliferation of the cells, which is important for tissue formation.

The investigators have discovered that there is a need to prepare coatings that promote enhanced cell attachment and proliferation of cells on the coated surface, particularly of endothelial cells and fibroblasts and have discovered novel and inventive coatings that achieve these results. These cell types are useful for enhancing tissue growth around an implant.

### SUMMARY

The present invention is directed to articles having biocompatible cell attachment coatings, and methods for forming these coatings. The coatings have a unique arrangement of coating components including a sulfonated component with aryl ketone reactive groups, and a cell attachment component that is a cell attachment molecule comprising amino acids. The cell attachment molecule comprises an extracellular matrix (ECM) protein, or a peptide that includes an active portion of an ECM protein. The sulfonated component is bonded in the coating using the aryl ketone reactive groups resulting in the presentation of chemical groups which provide a distinct and improved cell attachment surface. Generally, the coatings promote enhanced attachment of cells on the coated surface of the article. In turn, this increases the number of proliferating cells on the device surface. In the body, these improved functional surfaces can enhance generation of tissue in association with the coated surface.

In some aspects, the coatings are formed on implantable medical articles, and these articles can be used in methods for the treatment of a medical condition. In the body this can promote the formation of tissue in association with the article. The enhanced cell attachment and proliferation improves integration of the implant in the body, and makes the implant more effective for medical use. The invention also contemplates the use of these coatings on cell culture articles and in vitro methods for enhancing cell attachment.

In one aspect, the invention provides an article comprising a biocompatible cell attachment coating. The coating includes an intermediate coated layer comprising a first component comprising a sulfonate group and a bonding group comprising an aryl ketone functional group, and the first component is immobilized in the coating via the bonding group. The first component can be a polymeric or a non-polymeric compound. The coating also includes a second coated layer comprising a cell attachment molecule comprises an ECM protein, or a peptide that includes an active portion of an ECM protein, which is also immobilized in the coating. The intermediate coated layer is positioned between the second coated layer and a surface of the article. In use, the second coated layer is or becomes the outermost layer in the coating.

The coating can be formed by a method in which a first composition including the first component comprising the sulfonate group and the bonding group is applied to a device surface. A second composition including the cell attachment molecule comprising amino acids, which is an ECM protein, or a peptide that includes an active portion of an ECM protein, is applied after the first composition is applied. The method also includes a step of irradiating the coating which can be performed after the first composition is applied, after the second composition is applied, or both. Irradiation of the bonding group causes its activation and covalent bonding to a target moiety and immobilization in the coating. The target moiety which reacts with the bonding group can be a component of the device surface, another first component, the component selected from the ECM protein, or the peptide that includes an active portion of an ECM protein. The presentation of components in the coating achieved by irradiation and bonding using the bonding groups provides a particularly favorable presentation of coating components that has been shown herein to enhance the attachment of cells to the device surface.

Cell attachment studies associated with the invention revealed that the inventive coatings promoted a greater level of cell attachment to the coated surfaces over coatings using collagen alone, or the sulfonated reagent alone. The enhanced cell attachment results were rather surprising considering that, in theory, the coating process should result in the sulfonated reagent being buried under the collagen layer.

The coatings of the present invention can be formed on implantable medical articles such as, but not limited to, hernia meshes, aneurysm devices, and prosthetic devices such as coronary stents. Following implantation of the coated article in the body, the coating promotes an increased level of attachment of cells, such as endothelial cells and/or fibroblasts. Over time, tissue is formed on or around the coated implantable article. The natural tissue response can enhance integration, function, and lifetime of the implanted article.

The coatings of the present invention can be also formed on a cell culture article. Cells cultured on the coated cell culture article can be used for various purposes including in vitro testing or diagnosis, drug discovery, or for the culturing of cells that are introduced into the body.

### BRIEF DESCRIPTION OF THE DRAWINGS

FIG. 1 is a graph of results from an in vitro endothelial cell attachment assay on plastic surfaces coated with photoderivitized-heparin alone or in combination with photoderivitized-collagen.

FIG. 2 is a graph of an in vitro endothelial cell attachment assay on plastic surfaces coated with a non-polymeric sulfonated photoreagent alone or in combination with photoderivitized-collagen.

FIG. 3 is a graph of an in vitro endothelial cell attachment assay on plastic surfaces coated with a synthetic sulfonated photoderivitized-polymer alone or in combination with photoderivitized-collagen.

FIG. 4 is a graph of results from an in vitro fibroblast cell attachment assay on polypropylene mesh substrates coated with a non-polymeric sulfonated photoreagent alone or in combination with photoderivitized-collagen.

FIGS. 5A and 5B are graphs of in vitro endothelial cell attachment assays on plastic surfaces coated with a synthetic sulfonated photoderivitized-polymer alone or in combination with photoderivitized-gelatin or photoderivitized-bovine collagen at 2 hour (5A) and three day (5B) time points.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Generally, the coatings of the present invention include at least an intermediate coated layer and a second coated layer. If there is no other coated layer between the second coated layer and the structural material of the article on which the coating is formed, the intermediate coated layer may also be referred to as the “first coated layer.” The intermediate coated layer has a first component that includes a sulfonate group as well as a bonding group. The bonding group includes an aryl ketone functional group, which is reacted to immobilize first component in the coating by covalent reaction with the device surface or a coating material. The first component can be a polymeric or a non-polymeric compound. The coating also includes a second coated layer comprising a cell attachment molecule comprising amino acids, which comprises an extracellular matrix (ECM) protein, or a peptide that includes an active portion of an ECM protein, which is also immobilized in the coating. The intermediate coated layer is positioned between the second coated layer and a surface of the article. The second coated layer is, or can become, the outer coated layer that contacts cells during use of the article.

The coatings were able to display enhanced endothelial cell attachment and growth on culture plates and biomaterials. The coatings were also able to display enhanced fibroblast attachment on synthetic implantable meshes. The endothelial cell and fibroblast attachment was improved over coatings made from either constituent component alone. The coatings also demonstrated a significant increase in cell proliferation after a few days of culturing.

The coatings and methods of the invention can be used to promote the formation of tissue in association with the coated surface of the article. In some aspects the process of tissue formation includes endothelialization. Endothelialization refers to the attachment and formation of a persistent layer of endothelial cells on the surface of an implanted medical device. These coatings can enhance the adherence of endothelial cells and the subsequent proliferation of these cells, which in turn leads to a well-formed and persistent endothelial cell layer. In the body, the endothelial cell coverage may also correlate with reduced proliferation of smooth muscle cells and extracellular matrix synthesis as promoted by binding of endothelial mitogens such as FGF-2, a reduced SMC IL-1 response, and/or anti-thrombotic effects. The cell responses promoted by the coating of the invention are desirable, as they can reduce the rate of undesirable tissue responses that would otherwise lead to problems with integration of the device in the body. An implantable medical article with the coating of the invention can be introduced into a mammal for the prophylaxis or treatment of a medical condition. The coatings promote the formation of a mature endothelium in association with the article surface following a period of implantation. Mature endothelial cells can modulate other cellular responses, such as the proliferation of SMCs.

Endothelial cells are very flat, have a central nucleus, are about 1-2 μm thick and about 10-20 μm in diameter. Blood vessels and lymphatics are lined by endothelial cells; the layer being called the endothelium. Endothelial cells form flat, pavement-like patterns on the inside of the vessels and at the junctions between cells there are overlapping regions which help to seal the vessel. Endothelial cells are selective filters which regulate the passage of gases, fluid and various molecules across their cell membranes. Endothelial cells play a key role in angiogenesis, the development of new blood vessels from pre-existing vessels. Therefore, the coatings of the invention can promote the formation of new endothelial cell-derived tissue, including the formation of new blood vessels in the area of the implanted device.

Fibroblasts synthesize the extracellular matrix proteins, which are the structural framework for animal tissues, and play a key role in wound healing. Fibroblasts secrete the precursors of all the components of the extracellular matrix, primarily the ground substance and a variety of fibers, and are the most common cells of connective tissue in animals. Fibroblasts are morphologically heterogeneous with diverse appearances depending on their location and activity. Fibroblasts are derived from the mesenchyme, and express the intermediate filament protein vimentin, a feature used as a marker to distinguish their mesodermal origin. Therefore, the coatings of the invention can promote the formation of new tissue derived from fibroblasts, including the formation of new extracellular matrix in the area of the implanted device.

Various types of implantable medical articles can include a coating of the invention, and can be implanted at a target location in the body to provide a therapeutic effect to a subject.

One class of implantable articles is those for wound and tissue defect treatments. Exemplary articles include hemostatic barriers; mesh and hernia plugs; patches, including uterine bleeding patches. Hernia meshes typically include a woven material made from a synthetic plastic-like material, such as polypropylene. Hernia meshes can be in the form of a patch which is placed in approximation to the tissue weakness, or in a hole in the tissue to effectively serve as a plug. In some embodiments, the mesh with coating can be soft and flexible to conform to tissue movement and placement at the target site. A coated mesh of the invention can be in hernia repair methods that involve tension-free or laparoscopic tension-free procedures. The coating on the mesh, in combination with the scaffolding structural feature of the woven material, provides an excellent surface for cell attachment and new tissue, which eventually incorporates the mesh into the area of mesh placement. Exemplary hernia meshes and medical processes for hernia repair are described in U.S. Pat. Nos. 4,769,038 (C. R. Bard), 5,569,273 (C. R. Bard), and 5,769,864 (Surgical Sense).

Mesh or non-mesh support implants including a coating of the invention can also be used in a procedure to correct a condition of the urogenital tract. Mesh implants are well known in the art for the treatment of conditions such as stress urinary incontinence and vaginal prolapse (see, for example, U.S. Pat. Nos. 5,836,315, 6,306,079, 6,689,047, and 7,083,637).

Another class of implantable articles is those for cardiovascular treatment. Exemplary implantable cardiovascular articles include vascular implants and grafts, grafts, vascular prostheses including stents, endoprosthesis, stent-graft (such as abdominal aortic aneurysms (AAA) stent-grafts), and endovascular-stent combinations; small diameter grafts, abdominal aortic aneurysm grafts; atrial septal defect (ASD) patches, patent foramen ovale (PFO) patches, ventricular septal defect (VSD) patches, pericardial patches, epicardial patches, and other generic cardiac patches; pericardial sacks; ASD, PFO, and VSD closure devices; mitral valve repair devices; heart valves, venous valves, aortic filters; venous filters; left atrial appendage filters; valve annuloplasty devices; implantable electrical leads, including pacemaker and implantable cardioverter defibrillator (ICD) leads; and cardiac sensors.

Other implantable devices include ophthalmic devices, such as intraocular lenses.

Other implantable devices include those for the treatment of aneurysms, such as flow diverters, neuroaneurysm patches; neuroaneurysm coils; and aneurysm exclusion devices.

Other exemplary devices include self-expandable septal, patent ductus arteriosus (PDA), and patent foramen ovale (PFO) occluders constructed from nitinol wire mesh and filled or associated with polyester fabric (available from, for example, AGA Medical, Golden Valley, Minn.).

A medical article having a cell attachment coating can also be prepared by assembling an article having two or more “parts” (for example, pieces of a medical article that can be put together to form the article) wherein at least one of the parts has a coating. All or a portion of the part of the medical article can have an coating with the intermediate and second coated layers as described herein. In this regard, the invention also contemplates parts of medical articles (for example, not the fully assembled article) that have a coating of the present invention.

The biocompatible cell attachment coating can also be formed on the surface of a cell culture vessel. A “cell culture vessel” is an example of a cell culture article and, as used herein, means a receptacle that can contain media for culturing a cell or tissue. The cell culture vessel may be glass or plastic. Preferably the plastic is non-cytotoxic. Exemplary cell culture vessels include, but are not limited to, single and multi-well plates, including 6 well and 12 well culture plates, and smaller welled culture plates such as 96, 384, and 1536 well plates, culture jars, culture dishes, petri dishes, culture flasks, culture plates, culture roller bottles, culture slides, including chambered and multi-chambered culture slides, culture tubes, coverslips, cups, spinner bottles, perfusion chambers, bioreactors, and fermenters.

Optionally, an implantable medical article or a cell culture vessel can be associated with a “nanofibrillar structure,” which refers to a mesh-like network of nanofibers. A nanofibrillar structure can be a cell culture article and can be included in any sort of cell culture apparatus wherein cell attachment is desired, or where a cell culture process is performed. In many cases an article includes a network of nanofibers in addition to one or more other non-nanofiber materials. For example, a nanofibrillar structure can include a network of nanofibers on a support, wherein the support is fabricated from a material that is different than the nanofibers. The biocompatible cell attachment coating can be formed on a surface of the nanofibers, or on another portion of the article.

The article or device on which the cell attachment coating is formed can include natural polymers, synthetic polymers, metals, and ceramics. In some cases, combinations of any of these general classes of materials can be used to form the article, such as an implantable medical device. These materials can be described as “structural materials” for the body member of the article, in other words, the materials that provide the article with its three-dimensional structure.

In some cases, one or more of the material(s) of the article or device can serve as a target for covalent bonding to the activated aryl ketone functional group. Such materials generally are a good source of abstractable hydrogens. Using these materials, the excited state of a photoactivated aryl ketone functional group can insert into a carbon-hydrogen bond by abstraction of a hydrogen atom from the structural material of the article, thus creating a radical pair. Subsequent collapse of the radical pair leads to formation of a new carbon-carbon bond, and covalent bonding of the first component of the coated layer to the structural material of the article.

Many plastic articles, such as those formed from synthetic polymers, can provide a good source of abstractable hydrogens. Exemplary synthetic polymers, such as oligomers, homopolymers, and copolymers resulting from addition, condensation, or ring opening polymerizations can be structural materials of the article. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, and vinylidene difluoride. Examples of condensation or ring-opened polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

Other plastic articles, such as those formed from halogenated polymers, for example, chlorinated and/or fluorinated polymers, may have surfaces that are poorly reactive or non-reactive with the activated aryl ketone functional group. If these surfaces are not modified, or if a reactive base coat is not a part of the coating, activation of the aryl ketone functional group can result in bonding between materials of the intermediate coated layer, which can be a better source of abstractable hydrogen atoms as compared to the substrate surface. In this case, for example, covalent bonds can be formed between the activated aryl ketone functional group and polymeric material in the intermediate coated layer.

Examples of polymers that provide a poorly reactive, or non-reactive surface include perfluoroalkoxy (PFA) polymers, such as Teflon™ and Neoflon™; polychlorotrifluoroethylene (PCTFE); fluorinated ethylene polymers (FEP), such as polymers of tetrafluoroethylene and hexafluoropropylene; poly(tetrafluoroethylene) (PTFE); and expanded poly(tetrafluoroethylene) (ePTFE).

Implantable articles that are formed from a metal or combination of metals generally have surfaces that are poorly reactive or non-reactive with the activated aryl ketone functional group. Generally, metal surfaces are chemically modified or provided with a base coat in embodiments wherein the aryl ketone functional group is desired to bond to or towards the article surface.

Metals that can be used to form the implantable article include platinum, gold, or tungsten, as well as other metals such as rhenium, palladium, rhodium, ruthenium, titanium, nickel, and alloys of these metals, such as stainless steel, titanium/nickel, nitinol alloys, cobalt chrome alloys, non-ferrous alloys, and platinum/iridium alloys. One exemplary alloy is MP35.

In some embodiments, if the structural material of the implantable article is poorly reactive or non-reactive with the activated aryl ketone functional group, a base coat can optionally be formed on the article surface. The base coat can be positioned between the article surface and the intermediate coated layer. Exemplary base coats include polymeric compounds such as Parylene™, or silane-containing compound, such as hydroxy- or chloro-silane.

Parylene™ (poly(para-xylylene) base layers are typically very thin (0.1 micron to 75 microns), continuous, inert, transparent, and conformal films. Parylene™ is applied to substrates in an evacuated deposition chamber by a process known as vapor deposition polymerization (VDP). This involves the spontaneous resublimation of a vapor that has been formed by heating di-para-xylylene, which is a white crystalline powder, at approximately 150° C., in a first reaction zone. The vapor resulting from this preliminary heating is then cleaved molecularly, or pyrolized, in a second zone at 650° C. to 700° C. to form para-xylylene, a very reactive monomer gas. This monomer gas is introduced to the deposition chamber, where it resublimates and polymerizes on substrates at room temperature and forms a transparent film. In the final stage, para-xylylene polymerizes spontaneously onto the surface of objects being coated. The coating grows as a conformal film (poly-para-xylylene) on all exposed substrate surfaces, edges and in crevices, at a predictable rate. Parylene™ formation is spontaneous, and no catalyst is necessary. A process for forming a Parylene™ base layer on the surface of a metal stent is described in detail in U.S. Publication No. 2005/0244453 (Nov. 3, 2005; Stucke et al.).

In one mode of practice, an optional base coat of Parylene is formed on the article surface, followed by disposing a composition that includes a first component comprising a sulfonate group and a bonding group comprising an aryl ketone functional group. The applied first component is then irradiated, which results in covalent bonding to the Parylene material.

The optional base coat can alternatively be a layer of hydrophilic polymeric material which passivates the device surface. A passivating base coat can be formed by using a hydrophilic polymer, such one formed from acrylamide, methacrylamide, acrylic acid, vinylpyrrolidone, or combinations thereof. A passivating hydrophilic polymer can also include pendent photoreactive groups so the polymer can be immobilized as a base coat layer on the device surface. One example of a hydrophilic passivating polymer is a photo-derivitized polyacrylamide polymer, the preparation of which is described U.S. Pat. No. 6,007,833, Examples 1 & 2.

Aryl ketone functional groups refer to those groups having the base structure of:

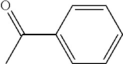

Aryl ketone functional groups include acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives can be used. Those functional groups containing two aryl groups can be referred to as diaryl ketone functional groups. Examples of aryl ketones include heterocyclic derivatives of anthrone, including acridone, xanthone, and thioxanthone, and their ring substituted derivatives. Some photoreactive groups include thioxanthone, and its derivatives, having excitation energies greater than about 360 nm.

These types of photoreactive groups, such as aryl ketones, are readily capable of undergoing the activation/inactivation/reactivation cycle described herein. Benzophenone is a particularly preferred photoreactive group, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (for example, carbon-hydrogen) is not available for bonding, the ultraviolet light-induced excitation of the benzophenone group is reversible and the molecule returns to ground state energy level upon removal of the energy source.

The first component comprising a sulfonate group and a bonding group comprising an aryl ketone functional group can be a polymeric compound or a non-polymeric compound. In some coatings, the first component is a polymeric compound. One or more sulfonate group(s) and one or more aryl ketone functional group(s) can be attached to the polymer as pendent groups. A “pendent group” is presented as a branch structure extending from a monomeric unit of the polymeric backbone. The pendent sulfonate group and/or aryl ketone functional group can be spaced away from the monomeric unit of the polymer backbone by spacer chemistry if desired.

In some aspects, the first component is a synthetic polymer. The synthetic polymer can include one or more hydrophilic monomeric units, and the overall property of the polymer can be water soluble. The hydrophilicity of a polymer can be described in terms of how soluble the polymer is in water. In some aspects, the polymer with pendent sulfonate and aryl ketone functional groups has a solubility in water of about 0.5 mg/mL or greater, about 1 mg/mL or greater, about 5 mg/mL or greater, or about 10 mg/mL or greater. Highly water-soluble polymers may have a solubility up to about 500 mg/mL or greater.

A polymer with pendent sulfonate and aryl ketone functional groups can be formed from one or more free radically polymerizable hydrophilic monomer(s). Examples of such monomers include, but are not limited to, acrylic monomers such as acrylic acid, methacrylate, methyl methacrylate, hydroxyethyl methacrylate, hydroxyethyl acrylate, methacrylic acid, acrylic acid, glycerol acrylate, glycerol methacrylate; and acrylamide-based monomers such as acrylamide, methacrylamide, aminopropylmethacrylamide, and derivatives and/or mixtures of any of these. Other hydrophilic monomers include, for example, methyl vinyl ether, maleic anhydride and vinyl pyrrolidone.

A polymer with pendent sulfonate and aryl ketone functional groups can be formed by any one of a variety of synthetic procedures. In one mode of synthesis, the polymer is formed by the free radical polymerization of a combination of monomers including a monomer bearing a sulfonate group and a monomer bearing an aryl ketone functional group. Optionally, a monomer that has neither a sulfonate nor an aryl ketone functional group can be included in the polymer. If an optional monomer that includes neither a sulfonate nor aryl ketone group is used, it can be present in the polymer in an amount of about 1% wt or greater, such as in the range of about 1% wt to about 50% wt, or about 2.5% wt to about 35% wt, or about 5% wt to about 30% wt.

Examples of monomers that include a sulfonate group that can be incorporated into the polymer to provide a sulfonate group pendent from the polymer include acrylamide-2-methylpropanesulfonic acid (AMPS), allyloxybenzenesulfonic acid (ABS), sodium methallyl sulfonate, and 3-allyloxy-2-hydroxy propane sulfonic acid. The monomers that includes a sulfonate group can be present in the polymer in exemplary ranges of about 10% wt or greater, 25% wt or greater, 40% wt or greater, 50% wt or greater, 60% wt or greater, 70% wt or greater, 80% wt or greater, 90% wt or greater, or 95% wt or greater, with exemplary ranges being from about 25% wt to about 95% wt, about 50% wt to about 95% wt, about 65% wt to about 95% wt, or about 70% wt to about 90% wt.

The amount of the sulfonate group can also be expressed in terms of molar quantity per gram of synthetic polymer. Exemplary ranges are from about 0.5 mmol/g to about 5 mmol/g, about 2.5 mmol/g to about 5 mmol/g, or about 4 mmol/g to about 5 mmol/g.

Exemplary monomers with photoreactive groups that can be incorporated into the polymer include those based on acrylamide and methacrylamide. One exemplary methacrylamide-based monomer with a pendent photoreactive groups is N-[3-(4-benzoylbenzamido)propyl]methacrylamide (BBA-APMA), the synthesis of which is described in Examples 1-3 of U.S. Pat. No. 5,858,653 (Duran et al.) Another exemplary methacrylamide-based monomer with a pendent photoreactive group is N-[3-(7-methyl-9-oxothioxanthene-3-carboxiamido)propyl]methacrylamide (MTA-APMA), the synthesis of which is described in Examples 1-2 of U.S. Pat. No. 6,156,345 (Chudzik et al.) The polymer with pendent sulfonate and aryl ketone functional groups is synthesized to have at least one aryl ketone functional group per polymer. More typically, the polymer is prepared to provide a loading of the aryl ketone functional groups on the polymer in the range of about 0.01 mmol/g to about 1 mmol/g (mmol aryl ketone group per gram of polymer), and more specifically in the range of about 0.1 mmol/g to about 0.5 mmol/g.

In another mode of synthesis, the polymer is formed by the free radical polymerization of a combination of monomers including a monomer bearing a sulfonate group and a monomer bearing a group that can be reacted with an aryl ketone-containing functional compound. For example, a copolymer comprising AMPS and N-(3-aminopropyl)methacrylamide (APMA) is first prepared. The copolymer is then reacted with the amine-reactive aryl ketone-containing functional compound 4-benzoylbenzoyl chloride under Schotten-Baumann conditions, such as described in U.S. Pat. No. 5,414,075 to provide pendent aryl ketone functional groups. The polymer can have a structure as shown below:

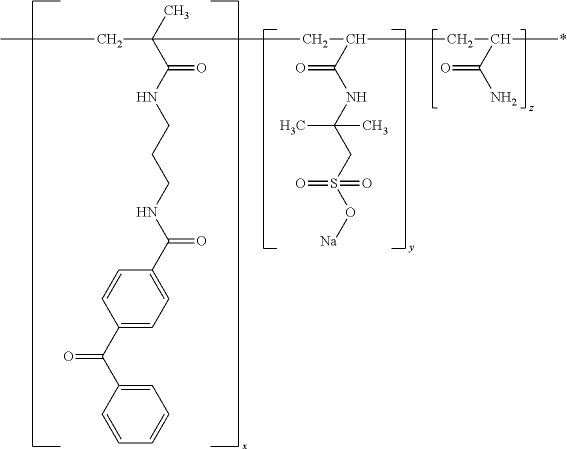

wherein x, y, and z independently represent the amount of each monomer species that is present in the polymer whether in random, block, or alternating configuration. Typically, x ranges from about 0.5 mole % to about 10 mole %, y ranges from about 25 mole % to about 95 mole %, and z range from about 5 mole % to about 70 mole %.

In some aspects, the polymerization mixture can include one or more monomer(s) that increase the polymer's solubility in polar protic solvents, such as alcohols like butanol, isopropanol, n-propanol, ethanol, and methanol, or polar aprotic solvents like acetone and ethyl acetate. Exemplary monomers include those having hydrophobic moieties such as dimethylacrylamides, diisopropylacrylamides, tert-butylacrylamides, and medium chain (e.g., C—C) alkyl acrylamides. A composition including the polymer and a polar protic or aprotic solvent can be useful for coating substrates formed from silicone or other polymers on which water does not sheet out well.

In other aspects, the intermediate coated layer includes a sulfonate-containing synthetic polymer that includes groups that can react with the ECM protein or peptide that includes an active portion of an ECM protein. For example, the sulfonate-containing synthetic polymer can include pendent amine-reactive groups, such as N-oxysuccinimide (NOS) groups. The NOS groups allows for bonding of an ECM protein, or a peptide that includes an active portion of an ECM protein. Other amine-reactive groups include, aldehyde, isothiocyanate, bromoacetyl, chloroacetyl, iodoacetyl, anhydride, isocyanate and maleimide groups.

A coating composition including a synthetic polymer comprising pendent sulfonate and aryl ketone functional groups can be prepared and applied to a substrate surface to form the intermediate coated layer. For polymers prepared predominantly or entirely of hydrophilic monomers, suitable polymer solvents include water, and other aqueous solutions Exemplary amounts of the synthetic polymer in the coating composition are in the range of about 1 mg/mL to about 50 mg/mL, or about 5 mg/mL to about 25 mg/mL. Increases in cell attachment can be promoted by increasing the sulfonate loading in the synthetic polymer, increasing the concentration of the synthetic polymer in the coating composition, or both.

In some coatings, the first component is derived from a natural polymer. In preferred modes of practice, the natural polymer is one that includes sulfonate groups (that have not been added to the polymer using a synthetic procedure). Exemplary sulfated natural polymers include monomeric or dimeric repeating units such as 2-O-sulfo-β-D-glucuronic acid, 2-O-sulfo-α-L-iduronic acid, 6-O-sulfo-β-D-galactose,/β-D-N-acetylgalactosamine-4-O-sulfate,/β-D-N-acetylgalactosamine-6-O-sulfate, β-D-N-acetylgalactosamine-4-O, 6-O-sulfate, α-D-N-sulfoglucosamine, and α-D-N-sulfoglucosamine-6-O-sulfate. Exemplary sulfonate-bearing natural polymers include heparin, heparan sulfate, keratan sulfate, chondroitin sulfate, and dermatan sulfate.

Photoderivatized heparin (“photoheparin”) was prepared as described in U.S. Pat. No. 5,563,056 (Swan et al., see Example 4), in which heparin was reacted with benzoyl-benzoyl-epsilon-aminocaproyl-N-oxysuccinimde (BBEAC-NOS) in dimethylsulfoxide/carbonate buffer. The solvent was evaporated and the photoheparin was dialyzed against water, lyophilized, and then dissolved in water. The structure of a portion of BBEAC-NOS-derivatized heparin is shown below.

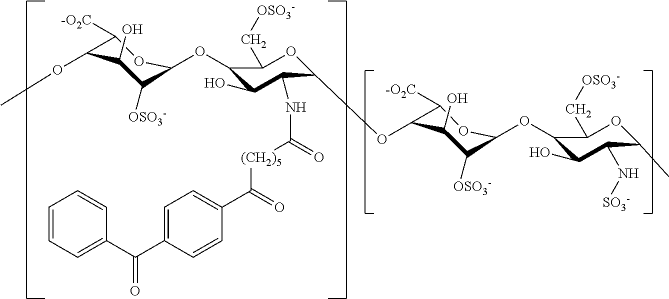

The amount of the sulfonate group can also be expressed in terms of molar quantity per gram of natural polymer. Exemplary ranges are from about 1.0 mmol/g to about 5.2 mmol/g, about 2.5 mmol/g to about 5.1 mmol/g, or about 4 mmol/g to about 5.1 mmol/g.

A coating composition including a sulfonate-bearing natural polymer derivatized with an aryl ketone functional group(s) can be prepared and applied to a substrate surface to form the intermediate coated layer. Suitable natural polymer solvents include water, and other aqueous solutions. Exemplary amounts of the natural polymer in the coating composition are in the range of about 1 mg/mL to about 50 mg/mL, or about 5 mg/mL to about 25 mg/mL.

In some aspects, the first component comprising a sulfonate group and a bonding group comprising a diaryl ketone functional group is a non-polymeric compound. In some cases, the non-polymeric compound has a nonpolymeric core molecule comprising an aromatic group, the core molecule having attached thereto, either directly or indirectly, one or more substituents with a sulfonate group, and two or more substituents with an aryl ketone group. Use of some non-polymeric compounds can provide an intermediate coated layer with a very high density of sulfonate groups, and can thereby allow the formation of a very thin intermediate coated layer.

In some aspects the non-polymeric compound has the formula:

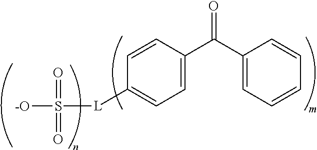

wherein L is a chemical group linking one or more sulfonate group(s) to one or more aryl ketone functional group(s), with n being an integer in the range of 1-2, and with m being an integer in the range of 1-3.

In the coating, T can be an atom or target moiety that is covalently bonded to the aryl ketone functional group.

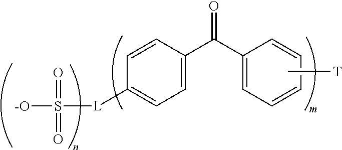

Such compounds include 4,5-bis(4-benzoylphenylmethyleneoxy)benzene-1,3-disulfonic acid or salt (compound A); 2,5-bis(4-benzoylphenylmethyleneoxy)benzene-1,4-disulfonic acid or salt (compound B); 2,5-bis(4-benzoylmethyleneoxy)benzene-1-sulfonic acid or salt (compound C); and N,N-bis[2-(4-benzoylbenzyloxy)ethyl]-2-aminoethanesulfonic acid or salt.

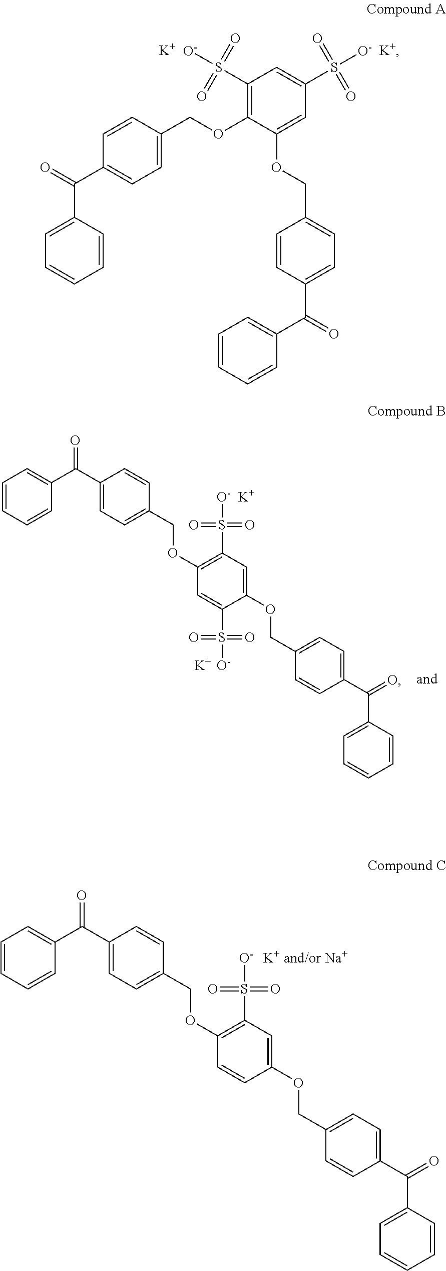

Non polymeric compounds having at least one sulfonate groups and two or more aryl ketone groups can be prepared in methods as described in U.S. Pat. No. 6,278,018.

The amount of the sulfonate group can also be expressed in terms of molar quantity per gram of non-polymeric compound. An exemplary range is from about 1 mmol/g to about 7 mmol/g, about 2.5 mmol/g to about 7 mmol/g, or about 4 mmol/g to about 6.5 mmol/g.

A coating composition including a non polymeric compound having pendent sulfonate and aryl ketone functional groups can be prepared and applied to a substrate surface to form the intermediate coated layer. Suitable solvents include water, and other aqueous buffers. Exemplary amounts of the non-polymeric compound in the coating composition are in the range of about 0.1 mg/mL to about 5 mg/mL, or about 0.25 mg/mL to about 1 mg/mL.

The coating process to form the intermediate coated layer can be performed using any one of a variety of methods. The coating method chosen can depend on one or more factors, such as the article that is coated, and/or the properties of the coating composition. In some modes of practice, the first component with the sulfonate group and aryl ketone functional group can be the primary component in the coating, and there may be no other, or insignificant amounts of other solid materials in the intermediate coated layer.

The coating process involves placing the coating materials in contact with the device surface, or device surface that has been pretreated with a base coat. In some modes of practice, the coating materials are applied to a surface and dried down, or partially dried down, and then the coating materials are irradiated. The process of applying can be performed using any one of a variety of techniques.

One exemplary method for applying the coating composition is by dip-coating. A typical dip-coating procedure involves immersing the article to be coated in the first coating composition, dwelling the object in the composition for a period of time (a standard time is generally less than about 30 seconds, and can even be less that 10 seconds in many cases), and then removing the article from the composition. After the article has been dip-coated in the coating solution, it is removed and dried, or partially dried. Drying can be carried out using any suitable method, including air-drying the dip coated article. Times up to 30 minutes can be sufficient to dry the coated article although shorter times may be also sufficient.

Other methods such as brushing, swabbing, or painting the first coating composition on the surface of the article can be performed to provide the intermediate coated layer. Alternatively, the first coating composition can be spray coated onto the surface of the article. An exemplary spray coating process and apparatus that can be used for coating implantable medical articles using the compositions of the present invention is described in U.S. Pat. No. 7,192,484 (Chappa et al.).

In other modes of practice, a liquid coating composition is applied to the article surface and then the article is irradiated while the liquid is in contact with the surface. In this mode of practice, the coating process is performed without drying down the coating solution on the surface of the substrate prior to the step of irradiating. For example, in solution, irradiation of cell culture articles can be performed by filling a well or the cell culture article with the liquid coating solution and then irradiating the well. In-solution coating can be performed to provide the intermediate coated layer to an implantable medical device as well. Such methods can provide a very thin intermediate coated layer of material on the article surface (see, for example, U.S. Pub No. 2010/0096320).

In some modes of practice, a step in the coating process involves irradiating the coating materials used to form the intermediate coating layer prior to applying the composition used to form the second coated layer. The process generally involves irradiation of the coating materials with UV light at a wavelength and amount in order to activate at least a portion of the aryl ketone functional groups to an excited state and cause their bonding to a target moiety. “Partial irradiation” involves irradiating the coating materials with a dose of irradiation so that a portion of the aryl ketone groups bond to target moieties, such as atoms of the device surface of other portions of components in the intermediate coated layer. Such irradiation can therefore result in “partial bonding” of the aryl ketone functional groups in the intermediate coated layer. One advantage of this approach is that the intermediate coated layer can be irradiated in a subsequent step to affect bonding of the unreacted aryl ketone groups to another target moiety. For example, after materials that form the second coated layer are applied on the intermediate coated layer, the coating can again be irradiated to cause bonding of the aryl ketone groups in the intermediate layer to the ECM protein or peptide that includes an active portion of an ECM protein, in the second coated layer. In this regard, reactive chemistries in the composition that forms the outer coated layer may not be required.

In other modes of practice, the materials of the intermediate coated layer are fully irradiated, meaning that all, or substantially all, of the aryl ketone functional groups are reacted and bonded to one or more target moieties.

Generally, aryl ketone functional groups are activated by UV radiation in the range of 330 nm to 340 nm. Light sources that provide output radiation sufficient to activate the photoreactive groups and promote formation of the coating can be used. Suitable light sources can incorporate, for example, metal halide bulbs, or other suitable bulbs that provide an activating source of irradiation. One suitable light source is a Dymax BlueWave™ Spot Cure System, which is commercially available from Dymax Corp. (Torrington, Conn.).

The amount of energy that is applied to the surface can vary depending on a number of factors, including the type and amount of polymeric or non-polymeric aryl ketone-containing compound used, the substrate material, and the type and amount of coating composition. In some aspects an amount of energy in the range of about 5 mJ/cm² to about 5000 mJ/cm² as measured at 335 nm, is applied to the surface; a more preferable range is from about 50 mJ/cm² to about 500 mJ/cm². Other ranges can be used in conjunction with the step of forming the coating. Partial bonding of the aryl ketone functional groups in the intermediate coated layer may be accomplished using amounts of energy in the lower ends of these ranges.

In one mode of practice, the coating is illuminated for 60 seconds using an ultraviolet Dymax™ Cure System at a distance of 20 cm. This distance and time can provided a coating with approximately 100 mJ/cm² in the wavelength range 330-340 nm.

The second coated layer of the article includes a cell attachment molecule comprising amino acids, which is an extracellular matrix (ECM) protein, or a peptide that includes an active portion of an ECM protein. As known in the art, ECM proteins provide structural support to cells and/or attach cells that reside in the ECM. Molecules on the surface of cells, such as integrins, carbohydrates, and other cell adhesion molecules can interact with ECM proteins to promote cell attachment. Exemplary ECM proteins include fibronectin, laminin, collagen, procollagen, elastin, vitronectin, tenascin, entactin, fibrinogen, thrombospondin, osteopontin (bone sialoprotein), osteocalcin, von Willibrand Factor, and active domains thereof.

An “active portion” (or “active domain”) of an ECM protein refers to an amino acid sequence found within the ECM protein that, in itself, provides function according to one or more properties of the ECM protein, such as providing structural support to cells and/or for attaching cells. The active portion may also be referred to as a “domain” or “motif.” The peptide that includes an active portion of an ECM protein can have a “core sequence” of amino acid residues, and optionally one or more additional amino acid residues that flank (i.e., on the C-terminus, N-terminus, or both) the core sequence. The one or more additional amino acids that flank the core sequence can correspond to the wild type ECM sequence in the relevant region of the protein, or can be an amino acid(s) that diverges from the wild type sequence (e.g., a “variant amino acid or sequence”). The variant amino acid or sequence can be one that enhances properties of the peptide, such as providing enhanced ligand interaction, and/or can facilitate formation of the second coated layer.

Active portions of ECM proteins are known in the art or can be determined using routine experimentation by carrying out assays that are commercially or described in a reference. For example, cell attachment assays which utilize peptides or proteins adhered to plastic or covalently immobilized on a support have been described and can be used to determine the activity of a desired peptide for promoting attachment of cells (see, for example, Malinda, K. M., et al. (1999) FASEB J. 13:53-62; or Kato, R., et al. (2006) J. Biosci. Bioeng. 101:485-95).

As used herein, a “peptide” is a short polymer of 25 or less amino acids linked by peptide bonds. As used herein, a “polypeptide” is a polymer of more than 25 amino acids linked by peptide bonds and which includes full length proteins. A peptide having an active portion of an ECM protein can be synthesized by solid phase peptide synthesis (SPPS) techniques using standard techniques, such as Fmoc synthesis. See, for example, Carpin, et al. (1970), J. Am. Chem. Soc. 92:5748-5749. Peptides described herein are also commercially available.

In one aspect of the invention type I collagen (collagen I) is present in the outer coated layer. Type I collagen is the most common of the collagens in vertebrates and makes up to 90% of the skeletons of the mammals, and also found in scar tissue, tendons, skin, artery walls, fibrocartilage, and bones and teeth. COL1A1 is the human gene that encodes collagen I, alpha 1 (1464 AA), with an accession reference number P02452 (C01A1_HUMAN) in UniProtKB/Swiss-Prot. The human sequence shares at least 90% sequence identity with, at least, chimpanzee (UPI0000E24950), dog (UPI0000EB21D9), and cow (P02453).

Type I procollagen is similar to other fibrillar collagens and has three polypeptide chains (α-chains) which form a unique triple-helical structure. It is a heterotrimer of two α1(I) and one α2(I) chains. Among species, the α1(I) chain is more conserved than the α2(I) chain (Kimura 1983). Type I collagen molecule contains an uninterrupted triple helix of approximately 300 nm in length and 1.5 nm in diameter flanked by short nonhelical telopeptides. The helical region is highly conserved among species (Chu et al. (1984) Nature 310:337-340).

Collagen peptides can also be used in the outer coating. Such peptides include RGD, YIGSR (SEQ ID NO:1), and (GPN1) repeats (see, for example, Johnson, G. (2000) J. Biomed. Mat. Res., 51:612-624). Collagen peptides, as well as other peptides that include a portion of an ECM protein, can be in linear or cyclic form (e.g., commercially available from Peptides International, Inc., Louisville, Ky.).

Recombinant collagen, such as recombinant human collagen, can be used to prepare the coatings. Recombinant collagen can be expressed in single cell organisms, such as yeast, in which collagen chains are expressed from a transgenic nucleic acid sequence. Recombinant human collagen I and human collagen III are commercially available (e.g., from FibroGen, Inc. San Francisco, Calif.), and can be prepared from human proalpha1(I), proalpha2(I) and both alpha and beta subunits of prolyl hydroxylase genes co-expressed in Pichia pastoris, and converted into mature collagen (from procollagen I) by proteinase digestion. Human proalpha1(III) can be expressed and digested in the same way to prepare mature collagen (from procollagen III).

Atelocollagen can be used to prepare the coatings. Atelocollagen can be prepared by removing antigenic telopeptides at each end of a collagen I molecule using a proteolytic enzyme, such as pepsin. Removal of the telopeptides generally improve solubility of the collagen, and render it soluble in an acidic solution (e.g., in the range of about 3.0 to 4.5) Atelocollagen can be prepared from collagen from an animal source, such as from porcine tissue. Methods for the preparation of atelocollagen are known in the art (see, for example, U.S. Pat. Nos. 3,949,073 and 4,592,864) and are also commercially available under the tradename Theracol™ (Regenerative Medical Systems, Hertfordshire, UK).

Hydrolyzed collagen (also known as gelatin) can also be used to prepare the coatings. Gelatin is formed from the hydrolysis of collagen using heat, and/or acid or alkali solutions, and results in collagen polypeptides or peptides that have a lower molecular weight than collagen. Recombinant gelatins having sizes of 100 kDa or 8.5 kDa are commercially available (e.g., from FibroGen, Inc. San Francisco, Calif.).

Peptides derived from a collagen sequence can also be used in the outer coating. Exemplary collagen peptides comprise the sequences DGEA (SEQ ID NO:2), KDGEA (SEQ ID NO:3), GER, and GFOGER (SEQ ID NO:4) (see, for example, Keely, P. J., and Parise, L. V. (1996) J Biol. Chem. 271:26668-26676; Kotite, N. J., and Cunningham, L. W. (1986) J Biol. Chem. 261:8342-8347; and Staatz, W. D., et al. (1991) J Biol. Chem. 266:7363-7367).

In some aspects of the invention the coating includes a laminin, or an active portion thereof. The laminin protein family includes multidomain glycoproteins that are naturally found in the basal lamina. Laminins are heterotrimers of three non-identical chains: one α, β, and γ chain that associate at the carboxy-termini into a coiled-coil structure to form a heterotrimeric molecule stabilized by disulfide linkages. Each laminin chain is a multidomain protein encoded by a distinct gene. Several isoforms of each chain have been described. Different alpha, beta, and gamma chain isoforms combine to give rise to different heterotrimeric laminin isoforms. Commonly used laminins are alpha 1, beta 1 and gamma 1 (i.e., Laminin-111) and alpha 5, beta 1 and gamma 1 (i.e., Laminin-511). Laminin sequences are available in UniProtKB/Swiss-Prot, including laminin subunit alpha-1 (P25391; LAMA1_HUMAN), laminin subunit alpha-5 (015230; LAMA5_HUMAN), laminin subunit beta-1 (P07942; LAMB1_HUMAN), and laminin subunit gamma-1 (P11047; LAMC1_HUMAN).

Peptides derived from a laminin sequence can also be used in the second coated layer. Exemplary laminin peptides comprise the sequences LRGDN (SEQ ID NO:5) and IKVAV (SEQ ID NO:6), YFQRYLI (SEQ ID NO:7) (Laminin A), YIGSR (SEQ ID NO:1), CDPGYIGSR (SEQ ID NO:8), and PDSGR (SEQ ID NO:9) (Laminin B1), and RNIAEIIKDA (SEQ ID NO:10) (Laminin B2). Synthetic peptides based on laminin sequences also include RQVFQVAYIIIKA (SEQ ID NO:11) and RKRLQVQLSIRT (SEQ ID NO:12) from the laminin alpha1 chain (Kikkawa, Y., et al. (2009) Biomaterials 30:6888-95; and Nomizu, M., et al. (1995) J Biol. Chem. 270:20583-90).

In some aspects of the invention, the second coated layer of the article includes a collagen or laminin polypeptide or peptide, or a peptide comprising a RGD motif. Preferred peptides are those containing RGD motifs such as the GRGDSP (SEQ ID NO:13) sequence from fibronectin as well as cell adhesive domains from collagen-I, collagen IV, and laminins I-III.

Fibronectin is a glycoprotein (˜440 kDa) that binds to integrins and has roles in cell adhesion, migration, differentiation, and growth. Fibronectin has accession number P02751 (FINC_HUMAN) in UniProtKB/Swiss-Prot.

The tripeptide Arg-Gly-Asp (RGD) is found in fibronectin as well as other proteins, and can mediate cell attachment. Certain integrins recognize the RGD motif within their ligands, and binding mediates cell-cell interactions. The RGD peptide and peptides that include the RGD motif can be used in the second coated layer. RGD-containing peptides include those having additional amino acid(s) that flank the core RGD sequence, such as RGDS (SEQ ID NO:14), RGDT (SEQ ID NO:15), GRGD (SEQ ID NO:16), GRGDS (SEQ ID NO:17), GRGDG (SEQ ID NO:18), GRGDSP (SEQ ID NO:13), GRGDSG (SEQ ID NO:19), GRGDNP (SEQ ID NO:20), GRGDSPK (SEQ ID NO:21), GRGDSY (SEQ ID NO:22), YRGDS (SEQ ID NO:23), YRGDG (SEQ ID NO:24), YGRGD (SEQ ID NO:25), CGRGDSY (SEQ ID NO:26), CGRGDSPK (SEQ ID NO:27), YAVTGRGDS (SEQ ID NO:28), RGDSPASSKP (SEQ ID NO:29), GRGDSPASSKG (SEQ ID NO:30), GCGYGRGDSPG (SEQ ID NO:31), GGGPHSRNGGGGGGRGDG (SEQ ID NO:32). In some cases the RGD-containing peptide has one or more lipophilic amino acid residues adjacent to the aspartic acid (D), such as RGDV (SEQ ID NO:33), RGDF (SEQ ID NO:34), GRGDF (SEQ ID NO:35), GRGDY (SEQ ID NO:36), GRGDVY (SEQ ID NO:37), and GRGDYPC (SEQ ID NO:38) (Lin, H. B., et al. (1994) J. Biomed. Mat. Res. 28:329-342). Peptides derived from fibronectin and that do not include an RGD motif, can also be used in the second coated layer. Other non-RGD peptides have or include sequences such as NGR, LDV, REDV (SEQ ID NO:39), EILDV (SEQ ID NO:40), or KQAGDV (SEQ ID NO:41).

Elastin (also knows as tropoelastin) is a component of elastic fibers, and includes a high amount of hydrophobic glycine and proline amino acids. Elastin has accession number P15502 (ELN_HUMAN) in UniProtKB/Swiss-Prot. Peptides derived from an elastin sequence can also be used in the second coated layer. Exemplary elastin peptides comprise the sequences VAPG (SEQ ID NO:42), VGVAPG (SEQ ID NO:43), VAVAPG (SEQ ID NO:44).

The ECM protein or peptide can also be modified with a reactive group which can provide further bonding within the coating. In some cases, the coating can be formed using photogroup-derivatized ECM protein, or a photogroup-derivatized peptide that includes a sequence derived from an ECM protein. The photo-derivation of collagen is used to exemplify the process, which can be used to photoderivatize other ECM proteins and peptides. Collagen, such as type I collagen, can be reacted with an amine reactive photogroup containing compound, such as BBA-EAC-NOS, which has a benzophenone photoactivatible group on one end (benzoyl benzoic acid, BBA), a spacer in the middle (epsilon aminocaproic acid, EAC), and an amine reactive thermochemical coupling group on the other end (N-oxysuccinimide, “NOS”). See U.S. Pat. No. 7,220,276.

In other cases, the ECM protein or peptide is coupled to a synthetic polymeric reagent that includes pendent photoreactive groups, and then this photopolymer-protein/peptide conjugate is used as a coating reagent. The synthetic polymer can be heterobifunctional and include a polymeric backbone with pendent photogroups and pendent thermochemically reactive groups which can react with an ECM protein or peptide to bond it to the polymer backbone (see, for example, U.S. Pat. No. 6,514,734; Clapper et al.). The heterobifunctional polymer can be prepared by the copolymerization of a base monomer, such as acrylamide or N-vinylpyrrolidone, with monomers having pendent photoreactive and/or thermochemically reactive groups. An exemplary thermochemically reactive group is N-oxysuccinimide (NOS) ester, which can react with an amine group on the ECM protein or peptide.

Alternatively, a monomer containing a polymerizable function (such as a vinyl group) and a NOS group is reacted with an ECM protein or peptide, and then this monomer is copolymerized with monomers containing photoreactive groups, and with a base monomer, such as acrylamide or N-vinylpyrrolidone. For example, a peptide monomer can be prepared by reacting a sulfhydryl group of the peptide with the maleimide group of N-[3-(6-maleimidylhexanamido)propyl]methacrylamide (Mal MAm). The peptide monomer is then copolymerized with acrylamide and a photoreactive methacrylamide monomer containing a substituted benzophenone (4-benzoylbenzoic acid, BBA). The photopolymer with pendent peptide molecules can then be used to form the second coated layer, which includes a step of UV irradiating the composition to bond the photogroups, thereby immobilizing the peptide via the polymer backbone.

In some coatings, type I collagen can be coated on the device to provide fibrillar or non-fibrillar collagen coated surfaces. In many aspects, the coating is formed by a method which provides collagen I in non-fibrillar form. For example photo-collagen-I can be prepared in a composition having a low pH (e.g., ˜pH 2.0) which is used to coat the surface of the implantable article, forming a coating that is non-fibrillar. Raising the pH of the solution (to, e.g., ˜pH 9.0) promotes the self-assemble into fibrils.

A coating composition including an ECM protein or peptide that includes an active portion of an ECM protein can be prepared and applied on the intermediate coated layer to form the second coated layer. In many aspects, the second coated layer is the outermost layer of the coating. Exemplary solvents for the polypeptide or peptide include, but are not limited to, water and other aqueous buffers. Exemplary amounts of the polypeptide or peptide in the coating composition are in the range of about 0.1 mg/mL to about 5 mg/mL, or about 0.25 mg/mL to about 1 mg/mL.

The coating can also be described in terms of the weight ratio of sulfonate groups to the cell attachment molecule (e.g., the ECM protein or peptide). In some aspects, coating comprises a mole to weight ratio of sulfonate groups to the cell attachment molecule in the range of about 0.5 mmol/g to about 7.5 mmol/g.

Irradiation of components of the second coated layer can be performed using conditions similar to irradiation of components of the intermediate coated layer. In one mode of practice, after the second coating composition is applied, the device is illuminated for 60 seconds using an ultraviolet Dymax™ Cure System at a distance of 20 cm.

In another aspect, the ECM protein or peptide that includes an active portion of an ECM protein, includes a pendent polymerizable group that can be reacted to form a polymerized second coated layer. In some aspects, a collagen macromer is used to form the second coated layer. A collagen macromer suitable for use in forming the present coatings is described in Example 12 of U.S. Pub. No. US-2006/0105012A1. Other macromers, such as laminin macromers, can be prepared using an analogous process.

Formation of the second coated layer including a macromer can be initiated by a polymerization initiator comprising a photogroup. Other agents that facilitate formation of a polymerized layer can be present in the composition. These can include, for example, polymerization accelerants which can improve the efficiency of polymerization. Examples of useful accelerants include N-vinyl compounds, particularly N-vinyl pyrrolidone and N-vinyl caprolactam. Such accelerants can be used, for instance, at a concentration of between about 0.01% and about 5%, and preferably between about 0.05% and about 0.5%, by weight, based on the volume of the coating composition.

As another option, after the second coated layer is formed, a temporary barrier or protective layer can be formed over the second coated layer. The barrier or protective layer can be formed from a degradable material that temporarily protects the second coated layer that includes the ECM protein or peptide that includes an active portion of an ECM protein. For example, the barrier layer can shield the coated article during the insertion process, but then degrades after the coated article is inserted into the body.

The coating can be prepared to have a desired thickness. In some aspects, the second coated layer has a thickness in the range of about 10 nm to about 100 nm. In some aspects, the coating has an overall thickness is in the range of about 20 nm to about 1 μm.

Cell attachment and proliferation can be measured in various ways. In vitro, cell attachment to a coated surface can be assessed using fluorometric methods. The indicator dye resazurin can be added to a cell culture vessel which is reduced by viable cells present into the highly fluorescent dye resorufin (579_{EX}/584_{EM}). Resazurin kits are commercially available, from, for example, Promega (CellTiter-Blue™). In vitro, cell proliferation can also be assessed using fluorometric methods. The water soluble dye MTT (3-(4,5-dimethylthiazol-2-yl)-2,5-diphenyltetrazolium bromide can be added to the cell culture vessel which is converted to insoluble formazan. The formazan is then solubilized and the concentration determined by optical density at 570 nm. MTT kits are commercially available, from, for example, Molecular Probes (Vybrant™).

In vivo, one way of observing cell attachment and proliferation for coated implantable articles is to histologically compare the surface of an article having a coating of the present invention with that of an article having an uncoated surface or having a chemically different coating. The histological comparison can be carried out after a time of implantation in a mammal. For example, in a test animal such as a rabbit histological examination can be carried out after a period of about 7 and/or a period of about 14 days. In a human subject, this period of time would correlate to about at least about two weeks, on average about four weeks, and in the range of about two weeks to about eight weeks.

Explanted samples can be examined using reagents that allow for the detection of cells associated with the surface of the coated article. In some methods of assessment, observation of endothelial cells is performed by treating the explanted article with BBI (bisbenzimide; Hoechst 33258). Observation of endothelial cells can also be performed by treating the explanted articles with Evans blue dye (Imai, H., et al. (1982) Arch Pathol Lab Med. 106:186-91).

The presence of endothelial cells can also be determined using antibodies to CD31, BS1 lectin, and factor VIII (Krasinski, K., et al. (2001) Circulation 104:1754). Antibodies against these proteins or lectins are commercially available, from, for example Calbiochem (San Diego, Calif.). In many cases, endothelial cells can be morphologically distinguished from other cell types such as certain immune cells. Smooth muscle cells can be distinguished from other cell types such as endothelial cells and fibroblasts using antibodies against actin (see, for example, Chamley, J. H., et al. (1977) Cell Tissue Res. 177:445-57).

Scanning electron microscopy can also be carried out to provide higher magnification of the surfaces of explanted article.

The surfaces of explanted articles can be scored according to endothelial cell coverage. The density of endothelial cells per unit area of the article can be performed. In some cases a scoring system can be employed to assess the level of endothelialization. For example, at a first level the article surface has essentially no cells; at a second level the article surface has some interspersed cells; at a third level the article surface has localized cell density in certain areas; at a fourth level the article surface has a consistent cell density covering most of the article; and at a fifth level the cell density is the highest and cell coverage masks the article.

The invention will be further described with reference to the following non-limiting Examples.

### Example 1

### Synthesis of Photocollagen

Bovine skin collagen (Semed S Powder) was purchased form Kensey Nash Corporation (Exton, Pa.). This collagen has the proportions of type I collagen (95%) and type III collagen (5%) that are usual for skin-derived collagens. Type 1 collagen was photoderivatized by the addition of (benzoylbenzoic acid)-(epsilon aminocaproic acid)-(N-oxysuccinimide)(BBA-EAC-NOS). BBA-EAC-NOS has a benzophenone photoactivatible group on one end (benzoyl benzoic acid, BBA), a spacer in the middle (epsilon aminocaproic acid, EAC), and an amine reactive thermochemical coupling group on the other end (N-oxysuccinimide, “NOS”). BBA-EAC was synthesized from 4-benzoylbenzoyl chloride and 6-aminocaproic acid. Then the NOS ester of BBA-EAC was synthesized by esterifying the carboxy group of BBA-EAC by carbodiimide activation with N-hydroxysuccimide to yield BBA-EAC-NOS. See U.S. Pat. Nos. 5,744,515 (columns 13 and 14), and 7,220,276. Atelocollagen (Biom'Up, Saint-Priest FRANCE) was photoderivitized in a similar manner.

### Example 2

### Synthesis of Photoheparin

Photoderivatized heparin (“photoheparin”) was prepared as described in U.S. Pat. No. 5,563,056 (Swan et al., see Example 4), in which heparin was reacted with benzoyl-benzoyl-epsilon-aminocaproyl-N-oxysuccinimde in dimethylsulfoxide/carbonate buffer. The solvent was evaporated and the photoheparin was dialyzed against water, lyophilized, and then dissolved in water.

### Example 3

### Synthesis of Sulfonate Photocrosslinker

Low molecular weight compounds that include photoreactive groups and sulfonate groups were used to form the coatings of the invention. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, which were prepared according to U.S. Pat. No. 6,278,018.

### Example 4

### Synthesis of Photopolyacrylamide AMPS

A photoderivatized-poly(acrylamide)-co-(2-acrylamido-2-methylpropanesulfonic acid) (photo-PA-AMPS) was prepared by a copolymerization of acrylamide, 2-acrylamide-2-methylpropanesulfonic acid (“AMPS”), and N-(3-aminopropyl) methacrylamide (“APMA”), followed by photoderivatization of the polymer using 4-benzoylbenzoyl chloride under Schotten-Baumann conditions according to U.S. Pat. No. 5,414,075. Polymers were prepared with 4% APMA-BBA and 30, 70 or 90% AMPS with 66, 26 or 6% acrylamide respectively. For these particular polymers the BBA was actually coupled to the APMA prior to polymerization. The monomers were then polymerized using AIBN and TEMED as initiators. The polymerization was carried out in degassed DMSO at 55-60 degrees C. for 16 hours and then dialyzed against water for 3 days and lyophilized.

### Example 5

### Synthesis of Photopolymer Peptides

Photopolymer peptides are prepared as described in U.S. Pat. No. 6,514,734 (Clapper et al.)

### Example 6

### Endothelial Cell Attachment with Photoheparin Coatings

Photocollagen and photoheparin were prepared as described in examples 1 and 2. 96 well polypropylene plates were coated with a photopolyacrylamide to passivate surfaces. A photopolyacrylamide polymer was used which contained 96.5% acrylamide and 3.5% APMA-BBA. Photopolyacrylamide was dissolved in water at 5 mg/mL and 200 uL was added to wells in 96 well plates. Plates were then exposed to UV light for 2 minutes and rinsed 3 times in water. Photoheparin coatings followed by photocollagen coatings were then applied to passivated plates as follows. Photoheparin was dissolved at 5, 10 or 25 mg/mL in sterile water and 100 uL was added to wells. Plates were then exposed to UV light for 1 minute and rinsed 3 times in water. Collagen was dissolved at 0.2 mg/mL in 12 mM hydrochloric acid (HCl) and 100 uL was added to wells. Plates were exposed to UV light for 1 minute and then rinsed 3 times in phosphate buffered saline (PBS) containing 1% Tween-20 followed by rinsing 2 times in PBS alone prior to running cell attachment assays.

Cell attachment assays were run using human coronary artery endothelial cell primary cultures available from Lonza. Cells were grown in endothelial basal media 2 (EBM-2) supplemented with endothelial growth medium 2 (EGM-2) singlequot supplements containing FBS and growth factors. Cells were trypsinized by washing 3 times in Dulbecco's PBS (DPBS, Invitrogen) and then incubating in 0.05% trypsin in EDTA (Invitrogen). Trypsinized cells were collected and neutralized with medium, rinsed one time in PBS and then resuspended in EGM-2 containing growth factors but not serum. Cells were resuspended at 175,000 cells per mL and 200 uL (35,000 cells) of cell suspensions were added to coated plates. Cells were incubated in plates for 2 hours and then rinsed 3 times in DPBS and then 200 uL of EGM-2 with serum was added to wells followed by 40 uL of Cell Titer Blue (Promega). Cells were incubated with Cell Titer Blue for 1 hour and then fluorescence was read with excitation at 560 nm and emission at 590 nm. A standard curve of HCAECs was prepared on tissue culture polystyrene plates at known concentrations of cells and the number of cells on coated plates was calculated using a linear fit of the standard curve. After determining the number of attached cells at 2 hours, cells were rinsed 3 times in PBS and then cultured in EGM-2 with serum for an additional 3 days and the number of attached cells was again determined using cell titer blue. 6 wells of the plate were used for each group and groups were prepared as outlined in Table 1 below.

TABLE 1
Cell attachment with Photoheparin

| | Photoheparin | Photocollagen |
|---|---|---|
| Treatment | (mg/mL) | (mg/mL) |
| A | 0 | 0 |
| B | 5 | 0 |
| C | 10 | 0 |
| D | 25 | 0 |
| E | 0 | 0.2 |
| F | 5 | 0.2 |
| G | 10 | 0.2 |
| H | 25 | 0.2 |

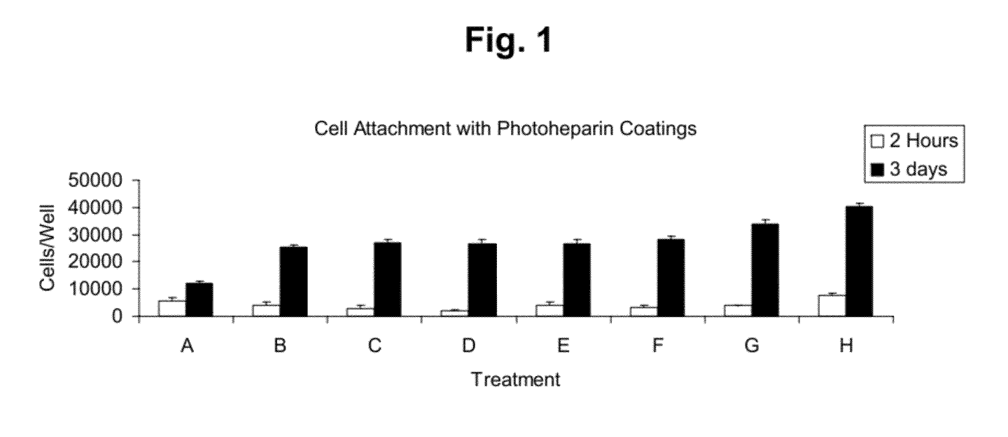

Cell attachment results at 2 hours and 3 days on coated plates are shown in FIG. 1. Results are shown as mean+/−SEM for n of 6 wells. Increased cell attachment was observed at 2 hours and 3 days when photocollagen was applied on top of a photoheparin coating. The degree of cell attachment increased with increasing concentrations of photoheparin. Additionally, photoheparin was found to increase cell attachment when used in the absence of photocollagen but to a lesser extent than the combination of the two coatings.

### Example 7

### Endothelial Cell Attachment with Sulfonate Photocrosslinker Coatings

Photocollagen and sulfonate photocrosslinker (4,5-bis(4-benzoylphenylmethyl-eneoxy)benzene-1,3-disulfonic acid) were prepared as described in examples 1 and 3. Additionally, a water soluble photocrosslinker containing amine groups (ethylenebis(4-benzoylbenzyldimethylammonium)dibromide; see example 2 of U.S. Pat. No. 5,714,360), as opposed to sulfonate groups, was used as a control. 96 well polypropylene plates were coated with a photopolyacrylamide to passivate surfaces. A photopolyacrylamide polymer was used which contained 96.5% acrylamide and 3.5% APMA-BBA. Photopolyacrylamide was dissolved in water at 5 mg/mL and 200 uL was added to wells in 96 well plates. Plates were then exposed to UV light for 2 minutes and rinsed 3 times in water. Photocrosslinker coatings followed by photocollagen coatings were then applied to passivated plates as follows. Sulfonate photocrosslinker or non-sulfonate photocrosslinkers were dissolved at 0.5 mg/mL in sterile water and 200 uL was added to wells. Plates were then exposed to UV light for 2 minutes and rinsed 3 times in water. Collagen was dissolved at 0.2 mg/mL in 12 mM hydrochloric acid (HCl) and 100 uL was added to wells. Plates were exposed to UV light for 1 minute and then rinsed 3 times in phosphate buffered saline PBS containing 1% Tween-20 followed by rinsing 3 times in PBS alone prior to running cell attachment assays. Two other additional experimental groups were run as well in which sulfonate and non-sulfonate photocrosslinkers at 0.5 mg/mL in water were mixed with photocollagen solutions to generate a solution of 0.25 mg/mL photocrosslinker and 0.1 mg/mL photocollagen in 6 mM HCl. 200 uL of this solution was added to plates and irradiated for 1 minute and then rinsed as described above. It was observed that when sulfonate photocrosslinker was combined with photocollagen aggregates of photocrosslinker and photocollagen formed and precipitated from solution.

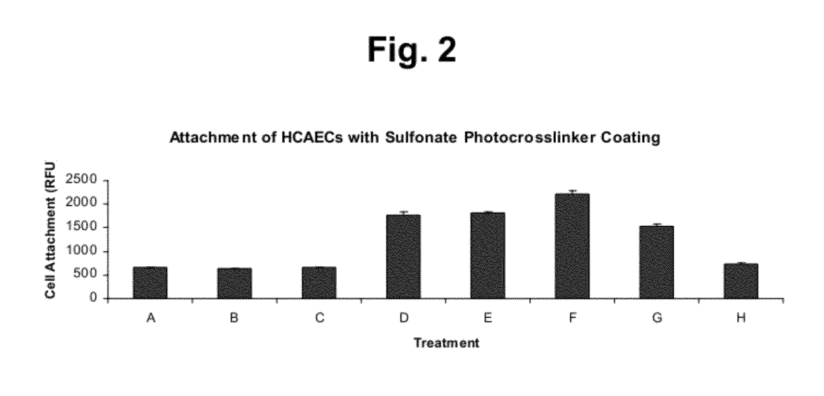

Cell attachment assays were run as described in Example 6 with the number of attached cells determined at 2 hours after addition to plates. In this experiment no standard curve was run, but rather Cell Titer Blue was used to determine a raw fluorescence value (RFU) for each treatment. Plates were coated as described in Table 2 below where the middle column describes the middle coating layer and the second column describes the top coating layer. Treatments G and H were coated as one combined coating layer of photocrosslinker and photocollagen. Cell attachment results are shown in FIG. 2. Results are shown as mean+/−SEM for n of 6 wells. Neither of the photocrosslinker coatings by themselves promoted cell attachment. However, when the sulfonate photocrosslinker was used prior to coating with photocollagen (Treatment F) enhanced attachment was observed compared to no photocrosslinker coatings (Treatment D) or non-sulfonate photocrosslinker containing coatings (Treatment E). When non-sulfonate photocrosslinker was coated in solution with photocollagen (Treatment G) no effect was seen as compared to photocollagen coatings alone. However the use of a sulfonate containing photocrosslinker in the same coating solution as photocollagen (Treatment H) resulted in reduced cell attachment as compared to photocollagen alone. This illustrates the distinct and unexpected benefits of applying the sulfonate containing coating layer prior to deposition of photocollagen which results in enhanced cell attachment.

TABLE 2
Cell attachment with Sulfonate Photocrosslinkers

| | | Photocollagen |
|---|---|---|
| Treatment | Photocrosslinker | (mg/mL) |
| A | None | 0 |
| B | 0.5 mg/mL Non-sulfonate | 0 |
| C | 0.5 mg/mL Sulfonate | 0 |
| D | None | 0.2 |
| E | 0.5 mg/mL Non-sulfonate | 0.2 |
| F | 0.5 mg/mL Sulfonate | 0.2 |

| | |
|---|---|
| G | Mixed solution with 0.25 mg/mL Non-Sulfonate and 0.1 |
| | mg/ml Photocollagen in 6 mM HCl |
| H | Mixed solution with 0.25 mg/mL Sulfonate and 0.1 |
| | mg/mL Photocollagen in 6 mM HCl |

### Example 8

### Endothelial Cell Attachment with Photopolyacrylamide AMPS Coatings

Photocollagen and photopolyacrylamide AMPS (PA-AMPS) were prepared as described in examples 1 and 4. PA-AMPS containing 30, 70 or 90% AMPS and 2% BBA on a molar basis was used. 96 well polypropylene plates were coated with a photopolyacrylamide to passivate surfaces. A photopolyacrylamide polymer was used which contained 96.5% acrylamide and 3.5% APMA-BBA. Photopolyacrylamide was dissolved in water at 5 mg/mL and 200 uL was added to wells in 96 well plates. Plates were then exposed to UV light for 2 minutes and rinsed 3 times in water. PA-AMPS coatings followed by photocollagen coatings were then applied to passivated plates as follows. PA-AMPS polymers were dissolved at 5 or 25 mg/mL in sterile water and 200 uL was added to wells. Plates were then exposed to UV light for 1 minute and rinsed 3 times in water. Collagen was dissolved at 0.2 mg/mL in 12 mM hydrochloric acid (HCl) and 100 uL was added to wells. Plates were exposed to UV light for 1 minute and then rinsed 3 times in phosphate buffered saline PBS containing 1% Tween-20 followed by rinsing 3 times in PBS alone prior to running cell attachment assays.

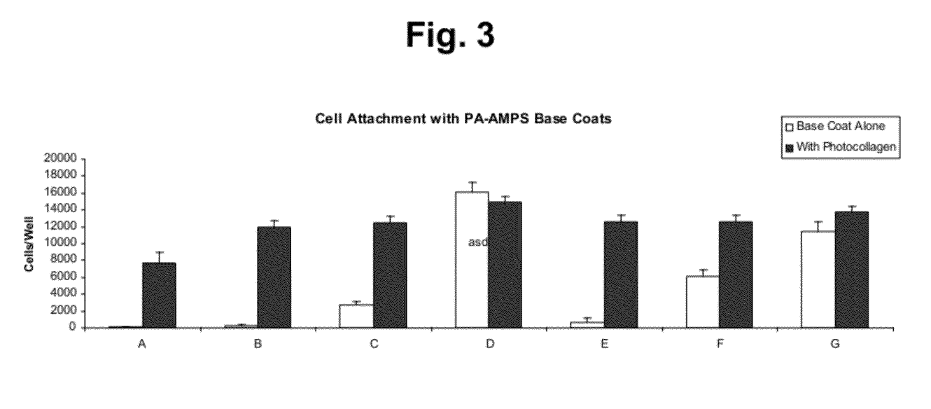

Cell attachment assays were run as described in Example 6 with the number of attached cells determined at 2 hours after addition to plates using a standard curve of known amounts of cells on TCPS. Treatments were as described in Table 3 below. Cell attachment results are shown in FIG. 3. Results are shown as mean+/−SEM for n of 6 wells. PA-AMPS coating by themselves promoted some degree of cell attachment and the amount of cell attachment was dependent on the polymer concentration and the % AMPS used in the polymer. When used in combination with photocollagen, higher levels of cell attachment were seen when PA-AMPS was used then when no PA-AMPS was used in the coating.

TABLE 3
Cell attachment with PA-AMPS

| | | PA-AMPS | |
|---|---|---|---|
| Treatment | % AMPS | (mg/mL) | Photocollagen |
| A | No PA-AMPS | 0 | PA-AMPS basecoats |
| B | 30 | 5 | were either left |
| C | 70 | 5 | uncoated or treated |
| D | 90 | 5 | with a topcoat of |
| E | 30 | 25 | photocollagen at 0.2 |
| F | 70 | 25 | mg/mL |
| G | 90 | 25 | |

### Example 9

### Coating with Sulfonate Base Coats and Photopolymer Peptide Top Coats

Coatings can be preparing a solution of photoheparin, sulfonate containing photocrosslinkers or PA-AMPS polymers in water or mixtures of water and isopropanol at concentrations between 0.5 and 50 mg/mL. This solution can be applied to a substrate and then immobilized through exposure to UV light. After exposure the substrate can be rinsed. Then a solution of photopolymer peptide (Prepared as in example 5) in water at a concentration of 0.1 to 5 mg/mL can be applied to the substrate and immobilized through exposure to UV light.

### Example 10

### Attachment of Human Dermal Fibroblasts to Polypropylene Mesh Coated with Sulfonate Photocrosslinker and Photocollagen

Polypropylene mesh substrates are available from multiple commercial sources including Davol (Warwick, R.I.), SurgicalMesh (Brookfield, Conn.), Kollsut Scientific Corporation (Pembroke Park, Fla.) and Ethicon (Somerville, N.J.). Sulfonate Photocrosslinker and photocollagen were prepared as described in examples 1 and 3. Mesh substrates were either left uncoated or coated with photocollagen alone or sulfonate photocrosslinker followed by photocollagen. Sulfonate photocrosslinker was prepared in water at 0.5 mg/mL and photocollagen was prepared in 12 mM HCl at 0.2 mg/mL and then filtered using 1 μm filter paper. Prior to coating, substrates were rinsed in isopropanol and then dried. The mesh was then dipped in the sulfonate photocrosslinker solution, slowly removed air dried and exposed to UV light for 1 minute. The mesh was then dipped in the photocollagen solution and slowly removed, air dried and exposed to UV light for 1 minute followed by rinsing in sterile deionized water.

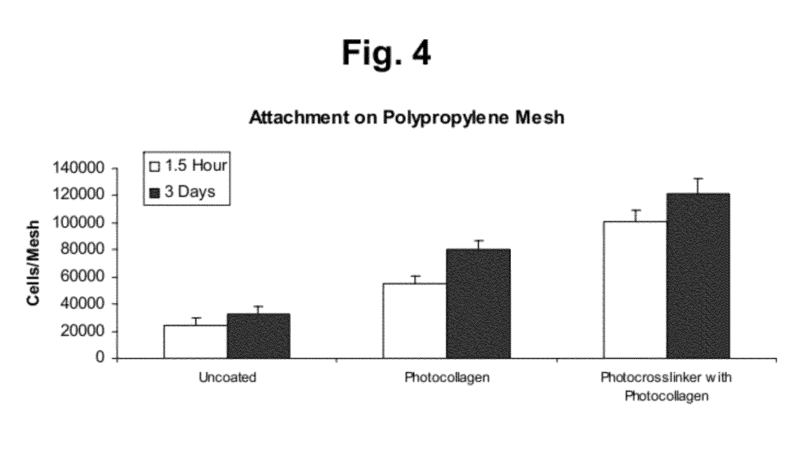

Cell attachment assays were performed using adult human dermal fibroblasts (HDFs) from Lonza cultured as per the manufacturer's instructions in FGM-2. Uncoated, photocollagen coated and sulfonate photocrosslinker/photocollagen coated meshes were placed into 2 mL cryovials and rinsed in DPBS. Cells were trypsinized and suspended in media at 375,000 cells/mL and 1.6 mL (600,000 cells) was added to the mesh in cryovials. The cryovials were rotated for 1 hour and then the mesh was placed into 12 well cell culture plates and the cells in the vial were poured over the mesh and incubated an additional 30 minutes. Cells were rinsed off the mesh using DPBS and the mesh was placed in fresh media. The number of cells attached to the mesh was determined using Cell Titer Blue and comparison to a standard curve of HDFs prepared in 12 well plates. The mesh was then rinsed 3 more times in DPBS and cultured in media for 3 more days at which point the number of cells attached to the mesh was determined again using cell titer blue. The results of cell attachment assays are shown in FIG. 4 as the mean+/−SEM for groups of 4. It can be seen that at both 1.5 hours and 3 days after addition of cells more cells are growing on photocollagen mesh as compared to uncoated mesh and that the use of a sulfonate photocrosslinker increases the number of attached cells over that seen with photocollagen alone.

### Example 11

### Endothelial Cell Attachment with Photogelatin Coatings

Recombinant human gelatin (8.5 kDa and 100 kDa) was purchased from FibroGen, Inc. (San Francisco, Calif.). Gelatin was photo derivatized using BBA-EAC-NOS as previously described in Example 1. Photopolyacrylamide AMPS was prepared as described in Example 4 with photopolyacrylamide AMPS prepared using 4% APMA-BBA and 96% AMPS. Bovine photocollagen was prepared as described in Example 1.

96 well polypropylene plates were coated with a passivating layer of photopolyacrylamide polymer as described in example 6. Photopolyacrylamide AMPS was dissolved at 5 or 25 mg/ml in water, filtered using a 5 μm syringe filter and then 100 μL of solution was added to plates. Plates were incubated 10 minutes and exposed to UV light for 1 minute followed by rinses in DI water. Photogelatin was dissolved in water at 0.2 mg/mL and photocollagen was dissolved in 12 mM HCl at 0.2 mg/mL. Both solutions were filtered with a 5 μm syringe filter and then 100 μL of solution was added to plates. Plates were incubated 10 minutes and exposed to UV light for 1 minute followed by rinses in PBS with 1% Tween-20.

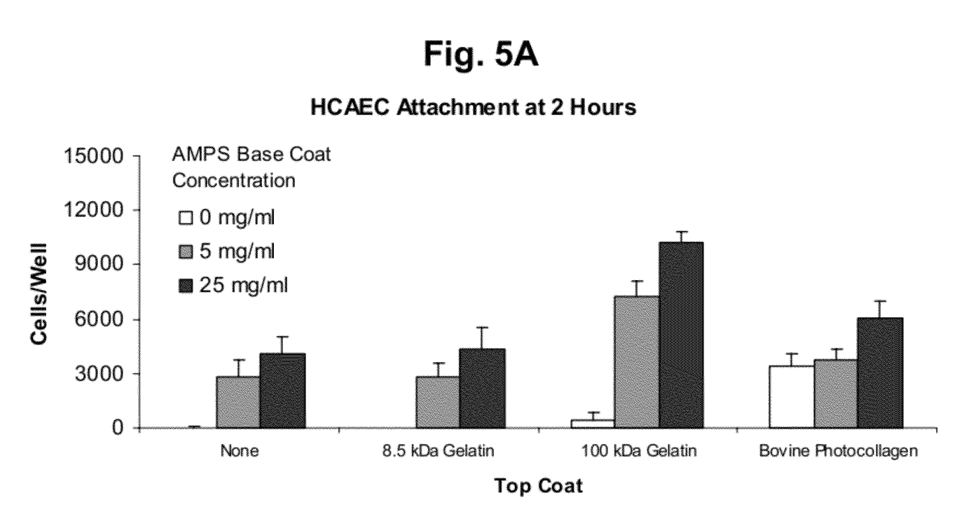

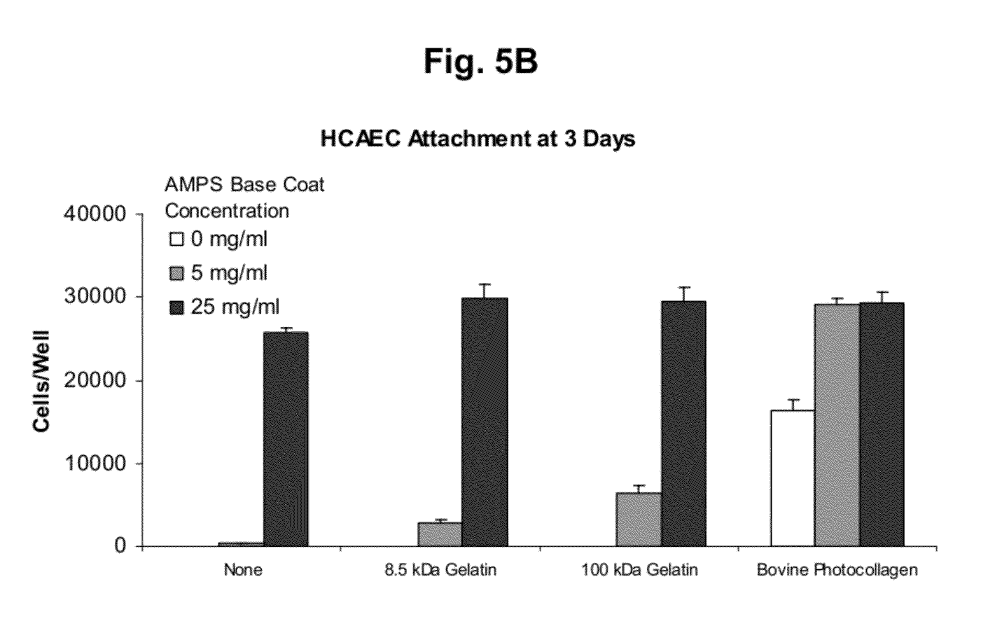

Cell attachment assays were run as described in Example 6 except that 20,000 endothelial cells were added to each well. The number of attached cells was determined after both the first 2 hour initial attachment and after 3 days in culture using cell titer blue and a cell standard curve. Results are shown for both initial attachment (FIG. 5A) and 3 day cell numbers (FIG. 5B). It can be seen that the number of attached cells with each topcoat increases with increasing concentration of the photopolyacrylamide AMPS polymer. This effect is observed for both initial attachment and at the 3 day timepoint. The highest concentrations of attached cells at the initial 2 hour timepoint was observed on the 25 mg/mL AMPS polymer basecoat with the 100 kDa photogelatin top coat. At 3 days, the highest numbers of attached cells were observed on the 8.5 and 100 kDa gelatin top coat with 25 mg/mL AMPS basecoat or on the photocollagen top coat with 5 or 25 mg/ml AMPS basecoats.

What is claimed is:

1. An article comprising a biocompatible cell attachment coating comprising:
(a) an intermediate coated layer comprising a first component comprising (i) a sulfonate group, (ii) a bonding group comprising an aryl ketone functional group; wherein the first component is immobilized in the coating via the bonding group; and
(b) a second coated layer comprising a cell attachment molecule comprising an extracellular matrix (ECM) protein, or a peptide that includes an active portion of an ECM protein that is immobilized in the coating;
wherein the intermediate coated layer is positioned between the second coated layer and a surface of the article.

2. The article of claim 1 wherein the extracellular matrix protein is selected from the group consisting of fibronectin, laminin, collagen, procollagen, elastin, vitronectin, tenascin, entactin, fibrinogen, thrombospondin, osteopontin (bone sialoprotein), osteocalcin, von Willebrand Factor, or the peptide includes an active portion of the recited ECM proteins for cell attachment.

3. The article of claim 2 comprising collagen or laminin, or a peptide comprising a RGD motif.

4. The article of claim 1, wherein the first component comprises a polymer backbone, and the sulfonate group and the aryl ketone functional group are pendent from the polymer backbone.

5. The article claim 4 wherein the polymer backbone comprises a polysaccharide.

6. The article of claim 5, wherein the polymer backbone comprises heparin which includes the following structure:

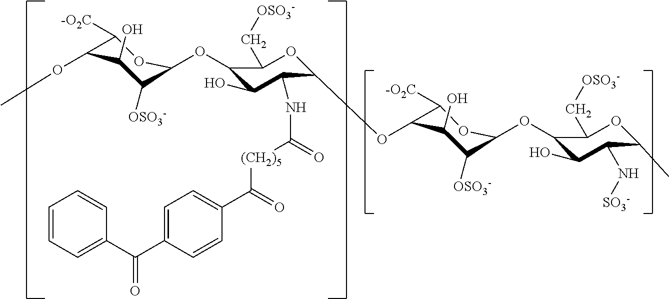

7. The article of claim 4, wherein the polymer backbone comprises a synthetic polymer

8. The article of 7, wherein the synthetic polymer comprises the following formula. 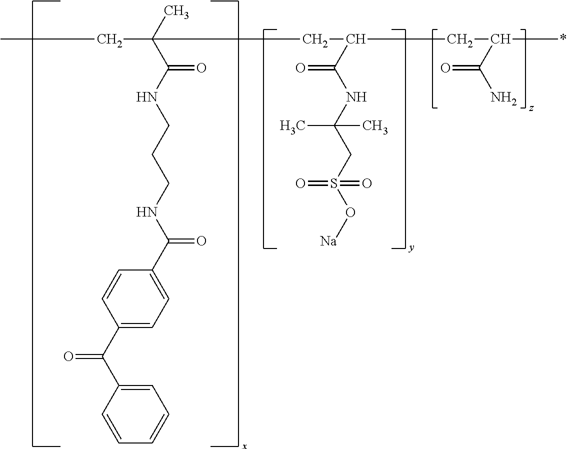
wherein x, y, and z independently represent the amount of each monomer species in random, block, or alternating configuration and x ranges from 0.5 mole % to 10 mole %, y ranges from 25 mole % to 98 mole %, and z range from 5 mole % to 70 mole %.

9. The article of claim 1 wherein the first component comprises the following formula: 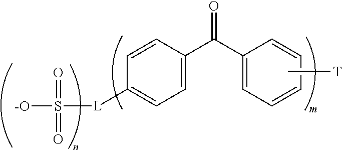
wherein L is a chemical group linking one or more sulfonate group(s) to one or more aryl ketone functional group(s), with n being an integer in the range of 1-2, and with m being an integer in the range of 1-3 and T is an atom or a target moiety that is covalently bonded to the aryl ketone functional group.

10. The article or method of claim 9 wherein the first component is selected from the group consisting of:

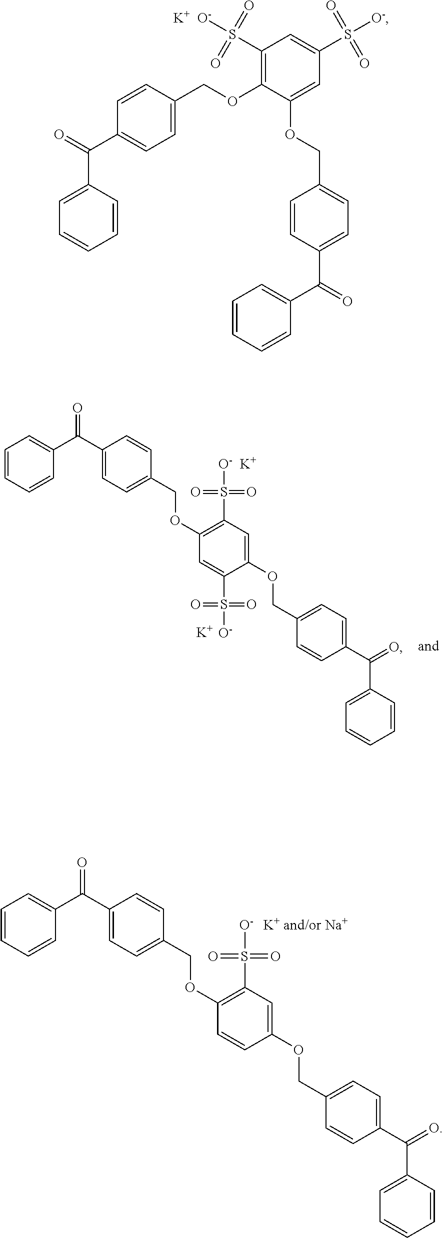

11. The article of claim 1, wherein the aryl ketone functional group is bonded to the surface of the medical article, to another first component in the coated layer, or both.

12. The article of claims 1, wherein the intermediate coated layer has a thickness in the range of 10 nm to 100 nm.

13. The article of claim 1, wherein the cell attachment molecule is covalently bonded to a target moiety selected from the first component, another matrix polypeptide, or both the first component and another matrix polypeptide.

14. The article of claim 1, wherein the cell attachment molecule comprises a pendent reacted aryl ketone group allowing the cell attachment molecule to be immobilized in the coating.

15. The article of claim 1, where, in the second layer, the cell attachment molecule is bonded to a synthetic polymer backbone, the synthetic polymer backbone further comprising a pendent reacted aryl ketone group allowing the cell attachment molecule to be immobilized in the coating.

16. The article of claim 1, wherein the coating comprises a mole to weight ratio of sulfonate groups to the cell attachment molecule in the range of about 0.5 mmol/g to about 7.5 mmol/g.

17. The article of claim 1, which is a medical article that is insertable or implantable into a portion of the body.

18. The article of claim 1, wherein the article is a cell culture article.

19. A method for forming a coated article comprising steps of:
(a) applying a first composition on an article surface, the first composition comprising first component comprising (i) a sulfonate group, (ii) a bonding group comprising an aryl ketone functional group;
(b) providing a second composition on the article surface, the second composition comprising a cell attachment molecule comprising an ECM protein, or a peptide that includes an active portion of an ECM protein for cell attachment;
(c) irradiating the device surface to cause bonding of the aryl functional group to a target moiety, wherein irradiating is performed after step (a), after step (b), or both after steps (a) and (b).

20. A method for promoting attachment and proliferation of cells to an article surface, the method comprising steps of
(a) providing an article comprising a cell attachment coating of claim 1;
(b) placing the article in an environment comprising cells, wherein the cells become attached to the coating.
